(19) United States
(12) Patent Application Publication
(10) Pub. No.: US 2004/0053329 A1
(43) Pub. Date: Mar. 18, 2004

(54) Endoglin-specific polypeptide, production and use thereof

(76) Inventors: Roland Kontermann, Ebsdorfergrund (DE); Daniel Miller, Dossenheim (DE); Rolf Muller, Marburg (DE)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/363,349

(22) PCT Filed: Sep. 4, 2001
(86) PCT No.: PCT/EP01/10197

(30) Foreign Application Priority Data
Sep. 4, 2000 (DE) 100 43 481.9

(51) Int. Cl. G01N 33/53; C07K 16/18; C07K 16/00; C12P 21/08; C12N 5/02; C12N 5/00; C12N 15/74; C12N 15/70; C12N 15/63; C12N 15/09; C12N 15/00; C12N 1/20; C12P 21/06

(52) U.S. Cl. 435/7.1; 530/388.25; 435/320.1; 435/69.1; 435/252.3; 435/325

(57) ABSTRACT

The present invention relates to a polypeptide which binds specifically to the extracellular domain of the human endoglin (CD105) protein, and also to its manufacture and use.

The present invention relates to a polypeptide which binds specifically to the extracellular domain of the human endoglin (CD105) protein, and also to its manufacture and use.

Vascular targeting, i.e. the selective recognition of cells or structures of the vascular bed is a relatively new concept in medicine. The aim is to cause certain diagnostically or therapeutically useful components to be transported specifically into the vascular bed. This approach finds application in tumour therapy, inter alia (Thorpe & Burrows, 1995, Breast Cancer Res. Treat. 36, 237-251). In the process, the tumour vascular bed is specifically attacked and eliminated, e.g. by means of a link with a cytotoxic component. This leads to an interruption in the supply of oxygen (hypoxia) and nutrients to the tumour tissue. The consequence is necrotisation of the tumour. The approach also finds applications in gene therapy, e.g. for the targeted transduction of endothelial cells with vectors used in gene therapy (e.g. viruses, liposomes, DNA-protein complexes) (Wickham et al., 1997, J. Virol. 71, 8221-8229).

A precondition for performing vascular targeting is to have ligands which recognise specific structures in the vascular bed. Examples of these are peptides or proteins which bind to particular receptors or other surface molecules on the endothelial cells. Examples of such receptors are the VEGF receptors or the αᵥ integrins (Burrows & Thorpe, 1994, Pharmac. Ther. 64, 155-174). In addition, antibody fragments which recognise specific structures in the vascular bed can also be used. Endoglin (CD105), for example, which is a member of the TGF-β family, is distinctly over-expressed by cells of the proliferating tumour endothelium (Miller et al., 1998, Int. J. Cancer 81, 568-572). Antibodies raised against endoglin have been described in the literature. The monoclonal antibody (MAb) SN6 was obtained by immunising mice with cell membranes of human leukaemia cells (Haruta & Seon, 1986, PNAS 83: 7898-7902). The MAb 44G4 was obtained by immunising mice with human pre-B leukaemia cells (Gougos & Letarte, 1988, J. Immunol. 141: 1925-1933). The MAb TEC4 and TEC11 were obtained by immunising mice with human umbilical cord endothelial cells (HUVEC) (WO 96/01653). The MAbs K4-2C10, D4-2G10, Y4-2F1 and P3-2G8 were obtained by immunising mice with purified human endoglin (WO 97/45450). All the antibodies directed against human endoglin known so far are thus derived from mice and as a rule they lead, in therapeutic applications in human beings, to the formation of human anti-mouse antibodies (HAMA), which in turn lead to the neutralisation of the therapeutic antibodies. The application of antibodies from the mouse or other organisms for therapeutic purposes is therefore very limited.

One object of the present invention is therefore to provide a polypeptide that binds specifically to CD105 and does not lead to the formation of neutralising HAMAs.

A further object of the present invention is to provide a polypeptide which is suitable for recruiting, for example, cytotoxic substances, liposomes or viruses on tumour endothelium.

In the present invention, it has now been surprisingly found that a polypeptide can be isolated which considerably improves the infection of human endothelial cells with an adenovirus.

The subject matter of the invention is therefore a polypeptide which binds specifically to the extracellular region of the human endoglin protein (CD105), the polypeptide containing one or more sequences according to SEQ ID No. 1. The extracellular region of the human endoglin protein comprises amino acids 1-559. Specific binding to human endoglin for the purposes of the invention is the case, for example, whenever the polypeptide is capable of precipitating endoglin from a cell suspension or detecting endoglin in an ELISA. The specific binding of a polypeptide of the invention is preferably determined by inhibition (=competition) of the binding of scFv C4, i.e. the binding is shown indirectly by means of the inhibition of a protein with the same binding characteristics. A polypeptide of the invention then binds specifically to endoglin if a 1000-fold molar surplus of the polypeptide relative to scFv C4 leads to the substantially complete inhibition of the binding of scFv to endoglin. A substantially complete inhibition of the binding preferably already occurs at a 100-fold molar surplus, more preferably at a 50-fold molar surplus. In a typical experiment to determine the specificity of the binding of a polypeptide of the invention, scFv C4 is used in a concentration of 1 μmol, and the polypeptide is added in different concentrations ranging between 1 μmol and 1 mmol. Alternatively, labelled polypeptides can be used.

In a further embodiment, the polypeptide additionally contains one or more sequences according to SEQ ID No. 2.

In one embodiment of the polypeptide of the invention, one to three cysteine residues, preferably one cysteine residue, are appended to the sequence according to SEQ ID No. 1 or to the sequences according to SEQ ID. Nos. 1 and 2 at each of the N and C termini. This residue serves to stabilise the polypeptide. In a preferred embodiment, a peptide linker is inserted between two sequences in each case. The peptide linker is preferably between approx. 12 and approx. 25 amino acids long.

In a preferred embodiment, the polypeptide which contains one or more sequences according to SEQ ID No. 1 contains one or more amino acid domains of a human antibody, these amino acid domains being selected from the framework region 1 (FR-1), FR-2, FR-3, FR-4, the complementarity determining region 1 (CDR-1) and/or CDR-2 of the antibody, preferably from FR-1 to FR-4, CDR-1 and/or CDR-2 the variable heavy chain (V_{H}). The framework regions of the variable light (V_{L}) or heavy chains have only slight sequence variability, and within the antibody they have a backbone function, by which the spatial structure is determined. The complementarity-determining regions have very high sequence variability within the variable domain of the light or heavy-chain regions. The structure of the CDRs (CDR-1, CDR-2 and CDR-3) determines the binding specificity of the antibody. In a preferred embodiment, the polypeptide of the present invention, in addition to at least one sequence according to SEQ ID No. 1, also contains FR-1 to FR-4, CDR-1 and CDR-2. It is particularly preferred for these regions to be selected from the V_{H}.

In a further embodiment, the polypeptide which contains one or more sequences according to SEQ ID No. 1 and SEQ ID No. 2 contains one or more amino acid domains of a human antibody, these amino acid domains being selected from the framework region 1 (FR-1), FR-2, FR-3, FR-4, the complementarity-determining region-1 (CDR-1) and/or CDR-2 of the antibody. Preferably, the amino acid sequence according to SEQ ID No. 1 is linked to the FR-1 to FR-4, CDR-1 and/or CDR-2 of the V_{H} and the amino acid sequence according to SEQ ID No. 2 is linked to the FR-1 to FR-4, CDR-1 and/or CDR-2 of the V_{L}, the SEQ ID No. 1 and SEQ ID No. 2 taking the position of the CDR-3 in the V_{H} and V_{L} respectively.

The term “human” for the purposes of the present invention refers to antibodies whose amino acid sequence exhibits a high degree of homology to the variable regions of the human heavy (V_{H}) and/or light chains (V_{L}) and which are therefore not immunogenic in humans, or only to a minor extent. A high degree of homology means that at least 80%, preferably 90%, particularly preferably 95%, and most preferably 98% of the amino acid residues are homologous. The degree of homology mentioned preferably applies to FR-1, FR-2, FR-3 and/or FR-4, while CDR-1 and CDR-2 domains do not exhibit any high degree of homology. Because of the low level of immunogenicity, the polypeptide of the invention, has some major advantages, precisely for therapeutic applications, compared to the murine antibodies described so far, since no neutralising antibodies are formed.

The degree of homology can be determined by means of a program such as ALIGN, for example, which is available on the Internet (e.g. under http://www.hgsc.bcm.tmc.edu/SearchLauncher/). Preferably, the mutations of the amino acid sequence are “conservative” changes, such as aspartic acid to glutamic acid, or leucine to isoleucine. The specific binding can be established by means of standard tests, such as ELISA.

In a further embodiment, a polypeptide for the purposes of the present invention contains not only FR-1, FR-2, FR-3, FR-4, CDR-1 and/or CDR-2, but also other components of an immunoglobulin, where these components can be of natural, partially synthetic or completely synthetic origin. Examples are components of the immunoglobulin isotypes, and parts of these immunoglobulins, such as the constant parts of the chain (C_{H} and/or C_{L}) or parts thereof. Depending on the other components added, the polypeptides of the invention can form Fab, F(ab¹)₂, “single chain Fv” (scFv), Fv dAb or Fd fragments.

The term “polypeptide” is used for amino acid chains of the invention with 9 or more amino acids. Polypeptides that have two or more identical binding sites, have an enhanced functional affinity (=binding strength) and are therefore preferred embodiments of the polypeptide of the invention. Polypeptides with an enhanced affinity for endoglin can be present, for example, in the form of a diabody (=scFv dimer) (Holliger et al., 1993, Proc. Natl. Acad. Sci. USA 90, 6444-6448), a single-chain multiple antigen-binding molecule (Brüsselbach et al., 1999, Tumour Targeting 4, 115-123) or a tandem scFv, or fused to dimerising regions of immunoglobulins, or dimerising peptides and regions of other proteins (Plückthun & Pack, 1997, Immunotechnology 3, 83-105).

A preferred embodiment of the polypeptide of the present invention is a polypeptide which contains one or more amino acid domains with a sequence according to SEQ ID No. 3. An amino acid domain for the purposes of the present invention is preferably between approx. 80 and approx. 150 amino acids long, and more preferably between approx. 100 and approx. 120 amino acids long, and contains not only a sequence according to SEQ ID No. 1, but also sequences of the human V_{L}-region.

In a further embodiment, the polypeptide of the invention contains one or more amino acid domains with a sequence according to SEQ ID No. 4.

In a particularly preferred embodiment, the polypeptide in each case contains at least one amino acid domain according to SEQ ID No. 3 and at least one according to SEQ ID No. 4.

In the polypeptide of the invention, there is preferably a peptide linker disposed between one or more amino acid domains according to SEQ ID No. 3 or SEQ ID No. 3 and SEQ ID No. 4, or between the one or more sequences according to SEQ ID No. 1 or SEQ ID No. 1 and SEQ ID No. 2. This peptide linker is preferably approx. 12 to approx. 25 amino acids long, especially 12 to 16 amino acids. The peptide linker serves to provide the spatial separation of the domains and/or sequences and facilitates the binding to endoglin. For separating the amino acid domains, a peptide linker with a sequence according to SEQ ID No. 5 is particularly suitable.

In order to facilitate the manufacture and isolation of the polypeptide, which is expressed recombinantly, for example, in a suitable cell, the polypeptide contains one or more secretion signals in a preferred embodiment. As a result of these secretion signals, the polypeptide of the invention is secreted by the cell into the periplasm and can be recovered directly from the culture medium of the production cell line. A particularly suitable secretion signal is the pelb secretion signal sequence (Lei et al., 1987, J. Bacteriol. 169, 4379-4383) with a sequence according to SEQ ID No. 6.

In a further embodiment, the secretion signal sequences can be cleaved off. This can be done, for example, by inserting peptide sequences which are recognised and cleaved by endopeptidases, or by inserting intein, for example. Cleaving off the secretion signal sequences can be advantageous whenever the secretion signal sequences are immunogenic in humans and the immunogenicity of the amino acid sequence of the invention is reduced by cleaving off the sequences.

A particularly preferred polypeptide contains a sequence according to SEQ ID No. 7. Here, the pelB secretion signal sequence is located at the N terminus, with the amino acid domain according to SEQ ID No. 3, the peptide linker according to SEQ ID No. 5 and the amino acid domain according to SEQ ID No. 4 arranged in the direction of the C terminus. In addition, the polypeptide also contains a hexahistidyl sequence (6×His-Tag) (Hochuli et al., 1988, Bio/Technol. 6, 1321-1325, Hoffmann & Roeder, 1991, Nucl. Acids Res., 19, 6337-6338), which makes it possible to purify the antibody via an Ni²⁺ affinity column, for example, and a peptide which is recognised by the anti-Myc antibody 9E10 (Munro & Pelham, 1986, Cell 46, 291-300).

A further subject matter of the present invention is a polypeptide which contains a variant of the amino acid sequence according to SEQ ID No. 1. One variant of the sequence according to SEQ ID No. 1 is sequences in which two amino acids are deleted, one amino acid is deleted and one amino acid is mutated, or two amino acids are mutated. Preferably here one mutation is, and more preferably both mutations are, “conservative” mutations. One speaks of “conservative” mutations when one amino acid is replaced by an amino acid of the same class. The different amino acid classes are amino acids with a non-polar, aliphatic side chain (Gly, Ala, Val, Leu, Ile and Pro), with polar, uncharged side chains (Ser, Thr, Cys, Met, Asn and Gln), with aromatic side chains (Phe, Tyr and Trp), with positively charged side chains (Lys, Arg and His) and with negatively charged side chains (Asp and Glu). Within one class, particularly preferred substitutions are those in which one amino acid is substituted for an amino acid with similar steric requirements, such as Ser for Thr or Gly for Ala.

Even more preferred, however, are variants of the amino acid sequence according to SEQ ID No. 1 in which only one amino acid is deleted or mutated. This mutation in the sequence according to SEQ ID No. 1 is preferably a conservative mutation.

The mutated and/or deleted polypeptides of the present invention are characterised in that they bind specifically to the extracellular domain of the endoglin. This specific binding can be detected in ELISAs against immobilised endoglin or by precipitation of endoglin by the peptides of the invention.

The polypeptides of the invention can also be present fused to at least one peptide and/or one protein. The term “peptide” refers to amino acid sequences of fewer than 50 amino acids, and “proteins” refers to amino acid sequences of 50 or more amino acids. A fusion is present whenever the amino acids of the polypeptide are linked to the peptide and/or the protein via a peptide bond. The fusion protein is preferably translated and encoded by an mRNA in one block. Suitable proteins and peptides are, for example, enzymes, growth factors, hormones, cytokines, chemokines, viral coat proteins, and/or antibodies. Fusion with a cytokine or chemokine permits the recruitment of a substance which is toxic for the target cell, for example, and thus makes possible the targeted lysis of tumour endothelium cells, for example. Fusion with a viral coat protein permits the manufacture of recombinant viruses bearing, on their surface, a polypeptide which is specific for endoglin and thus allows the recruitment of the respective recombinant virus to endothelial cells. A suitable coat protein is, for example, the adenovirus fibre protein. A similar objective can also be achieved by fusion with an antibody, preferably an scFv fragment, if it binds specifically to a certain virus. Other suitable peptides or proteins are those which are recognised by viral surface molecules or an antibody.

In a preferred embodiment of the polypeptide of the invention, the protein or peptide binds specifically to a receptor. Specific binding can be detected on immobilised receptors, for example, with labelled polypeptides. Examples of labels known in the state of the art are radioactive labels or fluorescence labels. Examples of suitable receptors are receptors which are present on cells of the immune system, such as CD3, CD4, CD8 CD28, F_{c}α-1 receptor, F_{c}γ-1, 2 or 3 receptor. These cells can be recruited by the interaction with endothelial cells.

A further subject matter of the present invention is a polypeptide which is coupled to at least one component. “Coupling” is understood to mean the covalent or non-covalent binding of one component to the polypeptide, where the polypeptide and the component are not translated together and are not encoded by an mRNA. Covalent coupling between the polypeptide of the invention and the component can be achieved by formaldehyde or glutaraldehyde, for example. Non-covalent coupling is obtained, for example, by incubation of a polypeptide of the invention fused to a peptide or protein that binds specifically to the knob domain of the adenoviral fibre proteins, together with adenovirus. Preferred components are peptides, proteins, enzymes, growth factors, hormones, cytokines, chemokines, viral coat proteins, carbohydrates, antibodies, lipids, isotopes, liposomes, viruses, virus-like particles, nucleic acids, and/or cells. The nucleic acids which are coupled to the polypeptide of the invention can be present in “naked” form or condensed with poly-lysine, for example.

The coupling of the polypeptide of the invention to liposomes is a particularly preferred embodiment of the present invention, because liposomes can be charged with a very wide variety of therapeutically active substances. Suitable liposomes are known from EP 0 555 333 or WO 00/74646, for example. Preferred liposomes are anionic liposomes which contain an anionic phospholipid in addition to cholesterol. The ratio between cholesterol and phospholipid in the liposome ranges between approx. 0.3 and approx. 1.2, preferably between approx. 0.4 and approx. 0.8. The coupling of the polypeptide of the invention to liposomes takes place, for example, via N-carboxyl phosphatidyl ethanol amine or glutaryl phosphatidyl ethanol amine.

The liposomes preferably contain at least one antisense RNA, at least one chemotherapeutic agent, at least one nucleic acid coding for an active agent or at least one active substance. If the liposomes contain nucleic acids, the liposome in a preferred embodiment additionally contains phosphatidyl ethanol amine (PEI), the PEI preferably being low-molecular-weight PEI with a molecular weight in the range of approx. 500 to approx. 25,000 Da, more preferably in the range of approx. 5,000 to 10,000 Da. The antisense RNA can, for example, inhibit the translation of genes which are needed for cell division. Chemotherapeutic agents comprise substances such as doxirubicin, cyclophosphamide, 5-fluorouracil, cis-platinum or taxol. The man skilled in the art is familiar with further chemotherapeutic agents which are used in tumour therapy and which are encompassed by the present invention. An active agent which is encoded by a nucleic acid contained in the liposomes can be an inhibitor of cell proliferation, for example. The man skilled in the art is familiar with suitable proteins, which encompass anti-oncogens, such as p53 or pRb, and cell cycle inhibitors, such as p21^{WAF}, p16^{INK}, p57^{INK2}, p27^{KIP} or GADD45. In addition, the nucleic acids can also code for cytostatic or cytotoxic proteins, such as perforin, granzyme, IL-2, IL-4, IL-12 or oncostatin M. An active substance can, for example, be any pharmacologically effective substance that is suitable for treating diseases in which endothelial cells are involved.

In a preferred embodiment of the polypeptide of the invention, the component binds specifically to a receptor. Specific binding can, for example, be detected on immobilised receptors with labelled polypeptides and/or labelled components. Examples of labels known in the state of the art are radioactive labels or fluorescence labels. Examples of suitable receptors are receptors which are present on cells of the immune system, such as CD3, CD4, CD8 CD28, F_{c}α-1 receptor, F_{c}γ-1, 2 or 3 receptor. These cells are recruited by the interaction of the component with one of the cell surface proteins and by the interaction of the polypeptide of the invention with endoglin to endothelial cells.

A further subject matter of the present invention is a nucleic acid which codes for a polypeptide of the invention. It is known that small changes in the sequence of a nucleic acid can be present, e.g. because of the degeneracy of the genetic code, or that untranslated sequences can be attached to the 5′ and/or 3′ end of the nucleic acid without changing the polypeptide encoded. This invention therefore also encompasses such “variants” of the nucleic acids described above.

“Variants” of the nucleic acids are understood to mean all nucleic acid sequences which are complementary to a nucleic acid sequence, which hybridise under stringent conditions to the reference sequence and which code for proteins that bind specifically to human endoglin.

“Stringent hybridisation conditions” are understood to mean those conditions under which hybridisation takes place at 60° C. in 2.5×SSC buffer, followed by several washing steps at 37° C. at a reduced buffer concentration, and remains stable.

In order to make it possible to introduce the above-mentioned nucleic acid and thus to allow the expression of the polypeptide in eukaryotic or prokaryotic cell by means of transfection, transformation or infection, the nucleic acid can be present as a plasmid, or as part of a viral, or non-viral vector.

A further subject matter of the present invention is therefore a vector, especially an expression vector containing a nucleic acid coding for a polypeptide of the invention. Particularly suitable viral vectors here are baculoviruses, vacciniaviruses, adenoviruses, adeno-associated viruses and herpes viruses. Particularly suitable non-viral vectors here are virosomes, liposomes, cationic lipids, or poly-lysine-conjugated DNA.

A further subject matter of the present invention is a cell containing at least one nucleic acid of the invention and/or at least one vector of the invention. Under conditions with which the man skilled in the art is familiar, and which lead to the activation of the regulatable elements used in each case, this cell expresses the polypeptide of the invention. The polypeptide can then be isolated from the cell or is secreted by the cell. For the recombinant production and subsequent purification of the expressed compounds of the invention, prokaryotic and eukaryotic cells are suitable, especially bacterial cells such as E. coli, yeast cells such as S. cerevisiae, insect cells such as Spodoptera frugiperda cells (Sf-9) or Trichoplusia ni cells, or mammalian such as COS cells or HeLa cells.

A further subject matter of the present invention is therefore a method of manufacturing a polypeptide of the invention in which at least one nucleic acid of the invention is expressed in a cell. If the polypeptide of the invention contains a cleavable secretion signal sequence, the latter can be cleaved off in a further step, such as by incubation with a suitable endopeptidase or, in the case of intein, by the addition of dithiothreitol (DTT) to the medium.

If a component is to be coupled to the polypeptide of the invention, said coupling can be effected by incubation or chemical reaction with at least one component. Coupling of this kind can already occur in the cell, but preferably only after purification of the polypeptide.

The polypeptide of the invention can be used as a diagnostic tool. A further subject matter of the present invention is thus the use of at least one polypeptide for detecting endoglin and/or endoglin-expressing cells or cell components in vitro and/or in vivo.

Detection can be achieved directly by fusion or coupling of a detectable component (e.g. with an enzyme or a radio-isotope) or indirectly by means of a labelled component which recognises the polypeptide of the invention. Preferred detection methods used are ELISA, RIA, immunofluorescence, immunoprecipitation or immunoscintillation.

The polypeptide of the invention directed against endoglin can also serve as a ligand, in order specifically to recognise and bind endoglin-expressing cells (e.g. tumour endothelium cells). A further subject matter of the present invention is thus the use of at least one polypeptide of the invention for binding to endoglin-expressing cells.

In this way, by means of the link to a second ligand by coupling or fusion, at least one peptide, at least one protein or at least one component can be recruited for endoglin-expressing cells. This second ligand can be an antibody molecule or fragment, a ligand for a cellular receptor, or a peptide that recognises a receptor on cells.

In a preferred use, the polypeptide of the invention has a cytotoxic effect on the endoglin-expressing cell. This effect is achieved, for example, by recruiting cytotoxic T-cells, or by fusion or coupling with cytokines or enzymes, such as “prodrug converting enzymes”.

In a further use of the polypeptide of the invention, the binding to the endoglin-expressing cell leads to the infection, transduction or transfection of the cell with a virus, a virus-like particle, a liposome, and/or a nucleic acid.

A further subject matter of the present invention is the use of at least one polypeptide, of at least one nucleic acid and/or of at least one vector as described above to treat diseases in which endothelial cells are involved. In a preferred embodiment, the polypeptides, nucleic acid and/or vectors of the invention are used to treat diseases which are characterised by the hyperproliferation of endoglin-expressing cells. Hyperproliferation of endothelial cells is observed, for example, in the neovascularisation of tumour tissue, which is why the treatment of tumour diseases is a particularly preferred use of the polypeptides, nucleic acids and/or vectors of the invention.

A further subject matter of the present invention is a pharmaceutical or diagnostic agent containing at least one polypeptide, at least one nucleic acid, and/or at least one vector as described above, and optionally suitable excipients and additives. Suitable excipients and additives lead, for example, to an improvement in the shelf life and to an improvement in the compatibility, or to an increase in the availability of the pharmaceutical or diagnostic agent of the invention, and the man skilled in the art is familiar with them.

The following illustration and the following examples are merely intended to describe the invention in more detail, and do not imply any limitation.

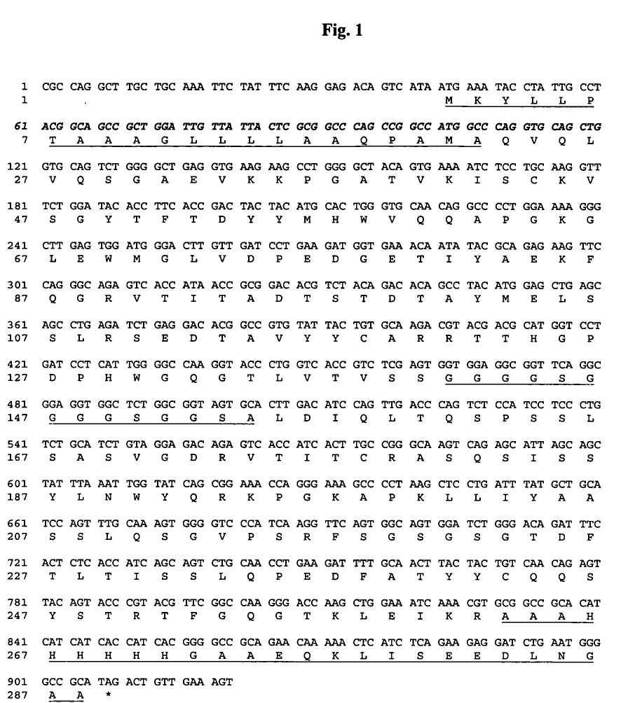

FIG. 1: The DNA sequence and protein sequence derived therefrom of the anti-endoglin polypeptide C4 of the invention in the form of an scFv fragment. The signal sequence, the linking peptide and the C-terminal sequences for purification and detection are underlined. The meaning of the individual nucleotide regions is as follows:

Nucleotides 1-42 5′ untranslated region

Nucleotides 43-106 DNA coding for pelB signal sequence (Lei et al., 1987, J. Bacteriol. 169, 4379-4383)

Nucleotides 107-465 DNA coding for human VH domain (semisynthetic consists of germ track V gene, and synthetic CDR3-FR4 region) (Griffiths et al., 1994, EMBO J. 13, 3245-3260)

Nucleotides 466-505 DNA coding for artificial peptide sequence (Huston et al., 1988)

Nucleotides 506-828 DNA coding for human VL domain (semisynthetic consists of germ track V gene, and synthetic CDR3-FR4 region) (Griffiths et al., 1994, EMBO J. 13, 3245-3260)

Nucleotides 829-837 DNA coding for artificial peptide sequence

Nucleotides 838-855 DNA coding for hexahistidyl sequence (Hochuli et al., 1988, Bio/Technol. 6, 1321-1325)

Nucleotides 856-864 DNA coding for artificial peptide sequence

Nucleotides 865-897 DNA coding for epitope of the anti-Myc antibody 9E10 (Munro & Pelham, 1986, Cell 46, 291-300)

Nucleotides 898-906 DNA coding for artificial peptide sequence

### EXAMPLES

### Example 1

Detection of Endoglin on Primary Endothelial Cells

The polypeptide scFv C4 shown in FIG. 1, which was isolated by phage display (Kontermann & Dübel 2000, Antibody Engineering, Springer Verlag), and which was present in the expression plasmid pHEN2 (MRC Centre for Protein Engineering, Cambridge, UK), was purified, after the induction of protein expression by the addition of isopropyl-β D-galactopyranoside (IPTG), from periplasmatic extracts of TG1 bacteria by means of immobilised metal affinity chromatography. For this purpose, for each litre of LB medium, which had been mixed with 100 μg/ml ampicillin and 0.1% glucose, 10 ml of an overnight culture of scFv C4 were added and shaken at 37° C. When an OD₆₀₀ of 0.8 was reached, there was an addition of 1 mM in an IPTG final concentration, and the bacteria were shaken for 3 hours at room temperature. The bacteria were centrifuged off, and the pellet was resuspended with extraction buffer (30 mM Tris-HCl pH 8, 1 mM EDTA, 20% saccharose). After incubation on ice for 15 min, MgCl₂ in a final concentration of 5 mM was added, and the solution was centrifuged again. The supernatant was dialysed against IMAC charging buffer (50 mM sodium phosphate buffer pH 7.5, 500 mM NaCl, 20 mM imidazole). The dialysate was loaded onto a Ni-NTA-charged column (Qiagen), which was equilibrated with charging buffer and washed with washing buffer (50 mM sodium phosphate buffer pH 7.5, 500 mM NaCl, 35 mM imidazole), and the bound antibody fragment was subsequently eluted with elution buffer (50 mM sodium phosphate buffer pH 7.5, 500 mM NaCl, 100 mM imidazole).

The purified polypeptide was used for the detection of endoglin. The bound polypeptide scFv C4 was detected indirectly in this process with the aid of monoclonal antibodies directed either against the hexahistidyl sequence or against the Myc epitope. The binding to purified endoglin was detected by means of ELISA. For this purpose, a polystyrene microtiter plate was coated with human endoglin (Konz. 10 μg/ml in PBS) overnight at 4° C. After a washing step in PBS, free binding sites were saturated by incubation with PBS, 2% skimmed milk powder. The anti-endoglin-antibody was adjusted in PBS, 2% skimmed milk powder, to a concentration of 50 μg/ml-5 ng/ml; 100 μl/well in each case were placed on the microtiter plate and incubated at room temperature for 1 hour. The plate was subsequently washed with PBS for 5 min. Bound antibody was detected with a peroxidase-labelled second antibody, which recognises the C-terminal Myc tag of scFv C4. The second antibody was adjusted to a concentration of 1 μg/ml in PBS, and 100 μl in each case were placed in each well of the microtiter plate. After incubation at room temperature for 1 hour, it was again washed with PBS for 5 minutes. Bound antibodies were detected by reacting the peroxidase substrate tetramethyl benzidine/H₂O₂. After the addition of 50 μl 1 M sulphuric acid, the colour change was determined in a photometer at a wavelength of 450 nm.

Endoglin from primary human umbilical cord endothelial cells (HUVEC) was detected by means of immunoprecipitation under non-denaturing conditions. For this purpose, the [³⁵S]-methionine-labelled endothelial cells were lysed with lysing buffer (10 mM Tris-HCl pH 7.5, 150 mM NaCl, 1% sodium deoxycholate, 1% Nonidet P40) for 30 min at 4° C. After an ultracentrifugation step at 40,000 r.p.m. for 20 min, the supernatant was mixed with 5 μg scFv C4, 5 μg of a negative control scFv or 5 μl of the murine anti-endoglin antibody SN6h (REF) and incubated for 1 hour at 4° C. This was followed by incubation with the anti-Myc antibody 9E10 (Munro & Pelham, 1986, Cell 46, 291-300) and then with A-sepharose protein, for 30 min at 4° C. in each case. The complexes were washed several times with lysing buffer and finally resuspended in 20 μL SDS-PAGE charge buffer. After separation in the SDS-polyacrylamide gel, the gel was immobilised for 30 min in 30% methanol and 10% acetic acid and subsequently mixed with amplification solution (Amersham-Buchler). The gel was dried and exposed with an x-ray film.

It became apparent that the scFv C4 polypeptide precipitated specifically to a band which was identical to that of the murine anti-endoglin antibody SN6h, whereas the same band could not be detected with the negative control antibody. It was thus possible specifically to detect endoglin in extracts of primary endothelial cells with scFv C4.

In further experiments, endoglin was detected on cells by means of immunofluorescence. For this purpose, various endothelial cells (HUVEC, HMVEC, HDMEC, HMEC) and non-endothelial cells (A549, HEK293) were incubated with scFv C4 at a concentration of 5-25 μg/ml or with the negative and positive control antibodies for 30 min at 4° C. After that, the recombinant polypeptides were incubated with the anti-Myc antibody 9E10 for 30 min at 4° C. Finally, all the batches were incubated with a Cy3-labelled anti-mouse antibody. The bound polypeptides were detected either by means of fluorescence microscopy or flow cytometry.

In these experiments, a specific fluorescence of endoglin-expressing endothelial cells could be detected, whereas various endoglin-negative cells did not exhibit any reaction. In the process, there was a typical surface staining of the cells, as was to be expected for a membrane protein.

### Example 2

A Bispecific Single-Chain Multi-Antigen-Binding Molecule for the Targeted Transduction of Endothelial Cells with Adenoviruses

The construction of a bispecific single-chain multi-antigen-binding molecule (reference is also made here to the patent applications DE 198 16 141 and EP 0 952 218), which is directed against endoglin and the knob domain of the fibre protein of adenoviruses of serotype 5, was performed on the DNA level as follows. For this purpose, the scFv fragment S11 was used (Watkins et al., 1996, Gene Ther. 4: 1004-1012). ScFv S11 binds to the knob domain of the fibre protein and neutralises wild-type infection through this binding. By means of polymerase chain reaction, sequences were appended to the V_{L} fragment of scFv S11 which code, at the 5′ end, for a BstEII restriction endonuclease cleavage site and a five-amino-acid-long binding peptide and, at the 3′ end, eight amino acids of the middle binding peptide and an AscI restriction endonuclease cleavage site. In the same way, sequences were appended to the V_{H} fragment of scFv S11 which code, at the 5′ end, for seven amino acids of the middle binding peptide and an AscI restriction endonuclease cleavage site and, at the 3′ end, for a SacI restriction endonuclease cleavage site and a five-amino-acid-long binding peptide. These fragments were cloned in the plasmid pAB1-scFv C4. The resulting bispecific single-chain multi-antigen-binding molecule (EDG-Ad) has the structure VHC4 peptide A-VLS11 peptide M-VHS11 peptide B-VLC4. Peptides A and B each have the sequence GGGGS, and peptide M has the sequence GGGGSGGRASGGGGGS. The monomeric molecule has a molecular weight of about 58 kDa and possesses one binding site each for endoglin and the knob domain. The bispecific single-chain multi-antigen-binding molecule was purified from the periplasm of induced bacteria, as described in Example 1. Binding studies showed that this molecule was fully functional. It recognised the knob domain in the ELISA and endoglin-expressing HUVEC in immunofluorescence.

In order to investigate adenoviral transduction, 2×10³ HUVECs or 3.5×10³ A549 cells were spread out on 96-well plates two days before they were infected with viruses. AdCMVLacZ, which expresses the lacZ gene under the control of the CMV promoter, was incubated for 1 hour at 37° C. with the bispecific single-chain multi-antigen-binding molecule EDG-Ad and subsequently added to the cells for 1 hour. As a control, viruses were used which were not incubated with EDG-Ad. The β-galactosidase was expressed by means of X-Gal staining. For this purpose, the cells were immobilised with 0.1% glutaraldehyde after a PBS washing step, washed again with PBS and then incubated in PBS at 37° C. with 0.8 mg/ml X-Gal, 3 mM K₃Fe(CN)₆, and 3 mM K₄Fe(CN)₆.

These experiments showed that adenoviruses alone exhibited only a very weak transduction with the virus titer used (8×10⁵ pfu). By complexing with EDG-Ad, however, this was increased significantly. This elevated, EDG-Ad-mediated transduction was dependent on the presence of endoglin on the target cells. Endoglin-negative cells (e.g. A549) were not therefore transduced to a greater extent. Furthermore, the EDG-Ad-mediated transduction of HUVEC was inhibited by pre-incubation with scFv C4. In contrast to this, the soluble knob domain, which inhibits the wild-type transduction completely by binding to the primary receptor (Coxsackie adenovirus receptor CAR), did not have any influence on the EDG-Ad-mediated transduction. Also pre-incubation with an RGD peptide, which inhibits the interaction of the adenoviral pentone base with the secondary receptor (αᵥ integrin) and in this way likewise prevents wild-type transduction, similarly had no influence on the EDG-Ad-mediated transduction. EDG-Ad-mediated transduction of endoglin-expressing cells is thus independent of the presence of the adenoviral receptors. On the contrary, EDG-Ad-mediated transduction occurs directly or indirectly via endoglin. The results prove that it is possible, with the aid of a bispecific molecule directed against endoglin and a viral coat protein, to recruit viruses to endoglin-expressing endothelial cells in a targeted way.

### Example 3

A Bispecific Single-Chain Multi-Antigen-Binding Molecule for the Targeted Lysis of Endothelial Cells by Cytotoxic T-Lymphocytes

The construction of a bispecific single-chain multi-antigen-binding molecule (reference is likewise made here to the patent applications DE 198 16 141 and EP 0 952 218), which is directed against endoglin and the ε-chain of the T-cell co-receptor CD3, was carried out on the DNA level as follows. For this purpose, scFv CD3v9 was used, which binds to the ε-chain of the T-cell co-receptor CD3. scFv CD3v9 is a humanised antibody fragment of the monoclonal antibody UCHT1 (Zhu & Carter, 1995, J. Immunol. 155: 1903-1910). By means of polymerase chain reaction, sequences were appended to the V_{L} fragment of scFv CD3 which code, at the 5′ end, for a BstEII restriction endonuclease cleavage site and a five-amino-acid-long binding peptide and, at the 3′ end, eight amino acids of the middle binding peptide and an AscI restriction endonuclease cleavage site. In the same way, CD3 sequences were appended to the V_{H} fragment of scFv sequences which code, at the 5′ end, for seven amino acids of the middle binding peptide and an AscI restriction endonuclease cleavage site and, at the 3′ end, for a SacI restriction endonuclease cleavage site and a five-amino-acid-long binding peptide. These fragments were cloned in the plasmid pAB1-scFv C4. The resulting bispecific single-chain multi-antigen-binding molecule (EDG-CD3) has the structure VHC4-peptide A-VLCD3 peptide M-VHCD3 peptide B-VLC4. Peptides A and B each have the sequence GGGGS, and peptide M has the sequence GGGGSGGRASGGGGGS. The monomeric molecule possesses one binding site each for endoglin and CD3. The bispecific single-chain multi-antigen-binding molecule was purified from the periplasm of induced bacteria, as described in Example 1. Binding studies showed that this molecule was fully functional. It recognised both endoglin-expressing HUVECs and CD3-expressing Jurkat cells in immunofluorescence.

In order to analyse an EDG-CD3-mediated cytolysis of endothelial cells by cytotoxic T-lymphocytes, europium-labelled HUVECs and isolated human T-lymphocytes which had been activated by phytohaemagglutinin and IL-2 were used. These cells were incubated in a ratio of HUVECs (target cell) to T-lymphocytes (effector) of 1:3, 1:10, 1:30 and 1:100 with different concentrations of EDG-CD3 (10 μg/ml to 1 ng/ml). After incubation for 4 hours in an incubator, the lysis of the endothelial cells was measured by means of time-resolution fluorescence. These results indicated an EDG-CD3-dependent cytolysis of the HUVECs. This was most pronounced at EDG-CD3-concentrations between 1-10 μg/ml and an effector-target-cell ratio of 100. Experiments with endoglin-negative control cells and the use of a bispecific single-chain multi-antigen-binding molecule directed against EDG and β-galactosidase (EDG-Gal) did not show any lysis of the endothelial cells. These experiments prove that EDG-CD3 is capable of recruiting T-cells to endoglin-expressing endothelial cells and triggering lysis of the cells in this way.

1. A polypeptide, which binds specifically to the extracellular domain of the human endoglin (CD105) protein, characterised in that the polypeptide contains one or more sequences according to SEQ ID No. 1.

2. The polypeptide as claimed in claim 1, characterised in that the polypeptide contains one or more sequences according to SEQ ID No. 2.

3. The polypeptide as claimed in claim 1, characterised in that the polypeptide contains one or more amino acid domains of a human antibody, said amino acid domains being selected from the framework region 1 (FR-1), FR-2, FR-3, FR-4, and/or the complementarity determining region 1 (CDR-1) and CDR-2 of the antibody.

4. The polypeptide as claimed in claim 1, characterised in that the polypeptide contains one or more amino acid domains with a sequence according to SEQ ID No. 3.

5. The polypeptide as claimed in claim 1, characterised in that the polypeptide contains one or more amino acid domains with a sequence according to SEQ ID No. 4.

6. The polypeptide as claimed in any of claims 3 to 5, characterised in that a peptide linker is disposed in each case between the amino acid domains.

7. The polypeptide as claimed in claim 6, characterised in that the peptide linker contains a sequence according to SEQ ID No. 5.

8. The polypeptide as claimed in claim 1, characterised in that the polypeptide contains one or more secretion signal sequences.

9. The polypeptide as claimed in claim 8, characterised in that the secretion signal sequence contains a sequence according to SEQ ID No. 6.

10. The polypeptide as claimed in claim 1, characterised in that the polypeptide contains one or more sequences according to SEQ ID No. 7.

11. The polypeptide as claimed in claim 1, characterised in that the polypeptide contains a variant of the SEQ ID No. 1.

12. The polypeptide as claimed in claim 1, characterised in that the polypeptide is fused to at least one peptide or protein.

13. The polypeptide as claimed in claim 12, characterised in that the protein or peptide is selected from the group consisting of an enzyme, a growth factor, a hormone, a cytokine, a chemokine, a viral coat protein, and an antibody.

14. The polypeptide as claimed in either of claims 12 or 13, characterised in that the protein or peptide is capable of binding specifically to a receptor.

15. The polypeptide as claimed in claim 1, characterised in that the polypeptide is coupled to at least one component.

16. The polypeptide as claimed in claim 15, characterised in that the component is selected from the group consisting of a peptide, a protein, an enzyme, a growth factor, a hormone, a cytokine, a chemokine, a viral coat protein, a carbohydrate, an antibody, a lipid, an isotope, a liposome, a virus, a virus-like particle, a nucleic acid, and/or a cell.

17. The polypeptide as claimed in claim 15 or 16, characterised in that the component is capable of binding specifically to a receptor.

18. The polypeptide as claimed in claim 17, characterised in that the liposome contains at least one antisense RNA, at least one nucleic acid coding for an active agent or at least one active substance.

19. The polypeptide as claimed in claim 18, characterised in that the active substance is a chemotherapeutic agent

20. A nucleic acid, characterised in that said nucleic acid codes for a polypeptide according to claim 1.

21. A vector, characterised in that said vector contains at least one nucleic acid according to claim 20.

22. A cell, characterised in that said cell contains at least one nucleic acid according to claim 20 and/or at least one vector according to claim 21.

23. A method of manufacturing a polypeptide according to claim 1, characterised in that at least one nucleic acid according to claim 20 is expressed in a cell.

24. The method as claimed in claim 23, characterised in that, in a further step, at least one component is coupled to the polypeptide.

25. The use of at least one polypeptide as claimed in claim 1 to detect endoglin and/or endoglin-expressing cells or cell components in vitro and/or in vivo.

26. The use as claimed in claim 25, characterised in that detection is carried out with an ELISA, a RIA, immunofluorescence, immunoprecipitation or immunoscintillation.

27. The use of at least one polypeptide as claimed in claim 1 for binding to endoglin-expressing cells, characterised in that the binding of the polypeptide has a cytotoxic effect on the endoglin-expressing cell.

28. The use of at least one polypeptide as claimed in claim 1, for the infection, transduction or transfection of endoglin-expressing cells.

29. The use of at least one polypeptide as claimed in claim 1, of at least one nucleic acid as claimed in claim 20 and/or of at least one vector as claimed in claim 21 for the production of a drug for the diagnosis and/or treatment of a disease which is characterised by the hyperproliferation of endoglin-expressing cells.

30. The use as claimed in claim 29, characterised in that the disease is a tumour disease.

31. A pharmaceutical or diagnostic agent containing at least one polypeptide as claimed in claim 1, at least one nucleic acid as claimed in 20, and/or at least one vector as claimed in claim 21 and optionally suitable excipients and additives.


SEQUENCE LISTING (14 sequences)

SEQ ID NO: 1 - protein, 9 aa, Artificial Sequence
  CDR3-H region
RTTHGPDPH

SEQ ID NO: 2 - protein, 9 aa, Artificial Sequence
  CDR3-L region
QQSYSTRTF

SEQ ID NO: 3 - protein, 120 aa, Artificial Sequence
  VH-domain consisting of semisynthetic germ line V-gene and synthetic CDR3-FR4 region
AQVQLVQSGAEVKKPGATVKISCKVSGYTFTDYYMHWVQQAPGKGLEWMGLVDPEDGETIYAEKFQGRVTITADTSTDTAYMELSSLRSEDTAVYYCARRTTHGPDPHWGQGTLVTVSSG

SEQ ID NO: 4 - protein, 107 aa, Artificial Sequence
  VL-domain consisting of semisynthetic germ line V-gene and synthetic CDR3-FR4 region
DIQLTQSPSSLSASVGDRVTITCRASQSISSYLNWYQRKPGKAPKLLIYAASSLQSGVPSRFSGSGSGTDFTLTISSLQPEDFATYYCQQSYSTRTFGQGTKLEIKR

SEQ ID NO: 5 - protein, 14 aa, Artificial Sequence
  linker peptide
GGGSGGGGSGGSAL

SEQ ID NO: 6 - protein, 21 aa, Pectobacterium carotovorum
  MISC_FEATURE: pe1B-signal sequence
MKYLLPTAAAGLLLLAAQPAM

SEQ ID NO: 7 - protein, 288 aa, Artificial Sequence
  anti-endoglin antibody fragment C4
MKYLLPTAAAGLLLLAAQPAMAQVQLVQSGAEVKKPGATVKISCKVSGYTFTDYYMHWVQQAPGKGLEWMGLVDPEDGETIYAEKFQGRVTITADTSTDTAYMELSSLRSEDTAVYYCARRTTHGPDPHWGQGTLVTVSSGGGGSGGGGSGGSALDIQLTQSPSSLSASVGDRVTITCRASQSISSYLNWYQRKPGKAPKLLIYAASSLQSGVPSRFSGSGSGTDFTLTISSLQPEDFATYYCQQSYSTRTFGQGTKLEIKRAAAHHHHHHGAAEQKLISEEDLNGAA

SEQ ID NO: 8 - DNA, 360 bp, Artificial Sequence
  DNA coding for VH-domain consisting of semisynthetic germ line V-gene and synthetic CDR3-FR4 region
gcccaggtgcagctggtgcagtctggggctgaggtgaagaagcctggggctacagtgaaaatctcctgcaaggtttctggatacaccttcaccgactactacatgcactgggtgcaacaggcccctggaaaagggcttgagtggatgggacttgttgatcctgaagatggtgaaacaatatacgcagagaagttccagggcagagtcaccataaccgcggacacgtctacagacacagcctacatggagctgagcagcctgagatctgaggacacggccgtgtattactgtgcaagacgtacgacgcatggtcctgatcctcattggggccaaggtaccctggtcaccgtctcgagtggt

SEQ ID NO: 9 - DNA, 321 bp, Artificial Sequence
  DNA coding for VL-domain consisting of semisynthetic germ line V-gene and synthetic CDR3-FR4 region
gacatccagttgacccagtctccatcctccctgtctgcatctgtaggagacagagtcaccatcacttgccgggcaagtcagagcattagcagctatttaaattggtatcagcggaaaccagggaaagcccctaagctcctgatttatgctgcatccagtttgcaaagtggggtcccatcaaggttcagtggcagtggatctgggacagatttcactctcaccatcagcagtctgcaacctgaagattttgcaacttactactgtcaacagagttacagtacccgtacgttcggccaagggaccaagctggaaatcaaacgt

SEQ ID NO: 10 - DNA, 42 bp, Artificial Sequence
  DNA coding for linker peptide
ggaggcggttcaggcggaggtggctctggcggtagtgcactt

SEQ ID NO: 11 - DNA, 63 bp, Pectobacterium carotovorum
  misc_feature: DNA coding for pelB-signal sequence
atgaaatacctattgcctacggcagccgctggattgttattactcgcggcccagccggccatg

SEQ ID NO: 12 - DNA, 921 bp, Artificial Sequence
  DNA coding for anti-endoglin antibody fragment C4
cgccaggcttgctgcaaattctatttcaaggagacagtcataatgaaatacctattgcctacggcagccgctggattgttattactcgcggcccagccggccatggcccaggtgcagctggtgcagtctggggctgaggtgaagaagcctggggctacagtgaaaatctcctgcaaggtttctggatacaccttcaccgactactacatgcactgggtgcaacaggcccctggaaaagggcttgagtggatgggacttgttgatcctgaagatggtgaaacaatatacgcagagaagttccagggcagagtcaccataaccgcggacacgtctacagacacagcctacatggagctgagcagcctgagatctgaggacacggccgtgtattactgtgcaagacgtacgacgcatggtcctgatcctcattggggccaaggtaccctggtcaccgtctcgagtggtggaggcggttcaggcggaggtggctctggcggtagtgcacttgacatccagttgacccagtctccatcctccctgtctgcatctgtaggagacagagtcaccatcacttgccgggcaagtcagagcattagcagctatttaaattggtatcagcggaaaccagggaaagcccctaagctcctgatttatgctgcatccagtttgcaaagtggggtcccatcaaggttcagtggcagtggatctgggacagatttcactctcaccatcagcagtctgcaacctgaagattttgcaacttactactgtcaacagagttacagtacccgtacgttcggccaagggaccaagctggaaatcaaacgtgcggccgcacatcatcatcaccatcacggggccgcagaacaaaaactcatctcagaagaggatctgaatggggccgcatagactgttgaaagt

SEQ ID NO: 13 - protein, 5 aa, Artificial Sequence
  Linker peptid A
GGGGS

SEQ ID NO: 14 - protein, 16 aa, Artificial Sequence
  Linker peptid M
GGGGSGGRASGGGGGS